(19) United States
(12) Patent Application Publication
(10) Pub. No.: US 2003/0170606 A1
(43) Pub. Date: Sep. 11, 2003

(54) High-throughput screening assays for modulators of mitochondrial membrane potential

(76) Inventors: Shu-Gui Huang, San Bruno, CA (US); Jin-Long Chen, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/567,856

(22) Filed: May 9, 2000

Related U.S. Application Data
(60) Provisional application No. 60/133,268, filed on May 10, 1999.

(51) Int. Cl. C12Q 1/00; G01N 33/53; G01N 33/567

(52) U.S. Cl. 435/4; 435/7.2

(57) ABSTRACT

The present invention provides methods for identifying modulators of uncoupling activity in mitochondria and for modulators of uncoupling proteins. In particular, this invention provides homogeneous assays for screening one or more test agents for the ability to modulate uncoupling activity in vivo.

### BACKGROUND OF THE INVENTION

The coupling between mitochondrial membrane potential and ATP production in eukaryotic cells is essential for survival. In the absence of such coupling, cells lack the ability to produce ATP, and, as a result, cannot sustain the metabolic processes necessary for life. Nevertheless, an ability to modulate the level of coupling in the mitochondria of cells would provide a valuable tool for modulating cellular metabolism, thereby providing a powerful treatment for metabolic conditions such as obesity.

In the absence of complete coupling, a substantial portion of the energy used by a cell is simply lost as heat. Such a “leak” in the energy pipeline within a cell in turn necessitates an increase in energy consumption in order to fulfill cellular energy requirements. Such an increase in energy consumption, e.g., by burning additional sources of energy such as fat, would have obvious benefit for treating conditions such as obesity.

Changes in mitochondrial membrane potential have also been implicated in cellular events such as apoptosis. Specifically, apoptosis is associated with a dramatic change in mitochondrial permeability, called the permeability transition (PT), which involves the formation of proteinaceous pores in the mitochondrial membranes. The PT causes a collapse of the mitochondrial membrane potential, which is a constant feature of apoptosis. See, e.g., Hirsch et al., (1997) Biosci. Rep. 17:67-76.

It has been discovered that multicellular organisms contain a class of proteins, called uncoupling proteins, that can mediate uncoupling activity in mitochondria. Originally identified several decades ago in the brown fat cells of hibernating animals such as bears, it is now recognized that humans have uncoupling proteins as well (see, e.g., Gura et al., (1998) Science 280:1369-70). Currently, at least five uncoupling proteins have been identified in humans, including UCP1 (see, e.g., Cassard et al., (1990) J Cell Biochem 43:255-64; UCP2 (see, e.g., Fleury et al., (1997) Nature Genet. 15:269-272), UCP3 (see, e.g., Boss et al., (1997) FEBS Lett. 408:39-42), UCP4 (see, e.g., Mao et al., (1999) FEBS Lett. 443:326-30), and BMCP1 (see, e.g., Sanchis et al., (1998) J. Biol. Chem. 273:34611-5). It has also been recognized that anywhere from 25% to 35% of the oxygen consumed by humans during food metabolism is lost due to uncoupling. Nevertheless, despite these advances, the precise role of these proteins, if any, in human uncoupling has not been established.

Several assays have been developed that are capable of detecting uncoupling protein activity, and have therefore proven useful in the study of uncoupling proteins and other compounds with potential uncoupling activity. For example, Tartaglia (U.S. Pat. No. 5,853,975) describe assays for uncoupling activity using flow cytometry to analyze fluorescent changes in cells. Such assays are limited, however, because they require the isolation or purification of the individual components of the assay, and are therefore inferior to homogeneous assays, which do not require such isolation or purification of assay components and therefore readily permit the use of high throughput screening methods.

Despite their clear advantages, no homogeneous assays have been developed that allow the detection of modulation of uncoupling activity in cells. As a result, few if any useful compounds with uncoupling activity, or with an ability to modulate uncoupling protein activity, have been identified. Thus, the art clearly lacks compounds that can facilitate uncoupling activity in a cell, e.g., by modulating the activity of uncoupling proteins, as well as methods to identify such compounds. The present invention fulfills these and other needs.

### SUMMARY OF THE INVENTION

The present invention provides novel methods for identifying modulators of uncoupling activity in mitochondria, e.g., by modulating the activity of uncoupling proteins in the mitochondria. In particular, this invention provides methods of screening one or more test agents for the ability to modulate uncoupling activity in vivo.

In one aspect, the present invention provides a method of screening a test agent for an ability to modulate the activity of an uncoupling protein, the method comprising: (i) expressing an uncoupling protein in a cell; (ii) introducing a fluorescent probe into the cell, wherein the fluorescence of the cell in the presence of the fluorescent probe is a function of the membrane potential (ΔΨm) in the mitochondria in the cell; (iii) contacting the cell with a test agent; and (iv) detecting the fluorescence of the cell; wherein an alteration in the fluorescence of the cell in the presence of the test agent compared to the fluorescence of the cell in the absence of the test agent indicates an ability of the test agent to modulate the activity of the uncoupling protein; and wherein the screening is performed in a homogeneous format.

In one embodiment, the method further comprises a secondary screening step, wherein the fluorescence of the cell in the presence of the uncoupling protein is compared to the fluorescence of the cell in the absence of the uncoupling protein; and wherein an ability of the test agent to modulate the fluorescence of the cell in the presence of the uncoupling protein, but not in the absence of the uncoupling protein, indicates that the activity of the test agent is specific for the uncoupling protein.

In one embodiment, the uncoupling protein comprises UCP1. In another embodiment, the uncoupling protein comprises UCP2. In another embodiment, the uncoupling protein comprises UCP3. In another embodiment, the uncoupling protein comprises UCP4. In another embodiment, the uncoupling protein comprises BMCP1. In another embodiment, the uncoupling protein is a hybrid protein comprising a heterologous polypeptide sequence that increases the localization of the protein to the mitochondrial membrane. In another embodiment, the heterologous polypeptide sequence is derived from the yeast ADP/ATP carrier (AAC) protein.

In another embodiment, the screening comprises high-throughput screening. In another embodiment, the high throughput screening comprises robotic high throughput screening. In another embodiment, the screening is performed using a multi-well plate. In another embodiment, the multi-well plate is a 96-well plate. In another embodiment, the multi-well plate is a 384-well plate.

In another embodiment, the cell is a yeast cell. In another embodiment, the yeast cell is Saccharomyces cerevisiae. In another embodiment, the yeast cell comprises an expression cassette comprising a polynucleotide encoding an uncoupling protein. In another embodiment, the method further comprises administering to the cell a permeabilizing agent. In another embodiment, the permeabilizing agent comprises zymolase. In another embodiment, the cell is selected from the group consisting of whole (untreated) cells, permeabilized cells, isolated mitochondria, and proteoliposomes reconstituted with a UCP.

In another embodiment, the fluorescent probe is DiSC3. In another embodiment, the fluorescent probe is a fluorescent dye other than DiOC6. In another embodiment, the alteration of fluorescence comprises an increase or decrease of at least about 30% in the fluorescence intensity in the presence of the test agent compared to the fluorescence intensity in the absence of the test agent.

In another aspect, the present invention provides a method for screening a test agent for the ability to modulate uncoupling activity in mitochondria, the method comprising (i) introducing a fluorescent probe into a cell, wherein the fluorescence of the probe in the cell is a function of the mitochondrial membrane potential (ΔΨm); (ii) contacting the cell with the test agent; and (iii) detecting the fluorescence of the cell; wherein an alteration in the fluorescence of the cell in the presence of the test agent compared to the fluorescence of the cell in the absence of the test agent indicates that the test agent is capable of modulating uncoupling activity in the cell; and wherein the screening is performed in a homogeneous format.

In one embodiment, the screening comprises high-throughput screening. In another embodiment, the high-throughput screening comprises robotic high-throughput screening. In another embodiment, the screening is performed using a multi-well plate. In another embodiment, the multi-well plate is a 96-well plate or a 384-well plate.

In another embodiment, the cell is a yeast cell. In another embodiment, the yeast cell is Saccharomyces cerevisiae. In another embodiment, the method comprises administering to the yeast cell a permeabilizing agent. In another embodiment, the permeabilizing agent comprises zymolyase. In another embodiment, the cell is selected from the group consisting of whole (untreated) cells, permeabilized cells, isolated mitochondria, and proteoliposomes.

In another embodiment, the fluorescent probe is DiSC3. In another embodiment, the fluorescent probe is a fluorescent dye other than DiOC6.

In another embodiment, the method further comprises expressing an uncoupling protein in the cell. In another embodiment, the uncoupling protein is UCP1. In another embodiment, the uncoupling protein is UCP2. In another embodiment, the uncoupling protein is UCP3. In another embodiment, the uncoupling protein is UCP4. In another embodiment, the uncoupling protein is BMCP1. In another embodiment, the cell comprises an expression cassette comprising a polynucleotide encoding the uncoupling protein.

In another embodiment, the method further comprises a secondary screening step, wherein the ability of the test agent to modulate uncoupling activity in the absence of the uncoupling protein is assessed, wherein an ability of the test agent to modulate uncoupling activity in a cell that is expressing the uncoupling protein, but not in a cell that is not expressing the uncoupling protein, indicates that the test agent is specific for the uncoupling protein. In another embodiment, the alteration in fluorescence comprises an increase or decrease of at least about 30% in the fluorescence intensity in the presence of the test agent compared to the fluorescence intensity in the absence of the test agent.

### BRIEF DESCRIPTION OF THE DRAWINGS

FIG. 1 illustrates (A) the permeabilization of yeast cells by zymolyase treatment and (B) an assay for the degree of permeabilization.

FIG. 2 illustrates the degree of permeabilization as assessed by substrate availability by glucose 6-phosphate dehydrogenase.

FIG. 3 shows the effects of a UCP1 activator (2-bromo-palmitate; “BrPalm”) and inhibitor (GDP) on the fluorescence of DiSC3 in permeabilized UCP1 cells.

FIG. 4 illustrates the use of K⁺ to assess the role of the plasma membrane in DiSC3 fluorescence.

FIG. 5 illustrates the influence of varying concentrations of KCl on DiSC3 fluorescence in permeabilized UCP1 cells.

FIG. 6 illustrates a method to differentiate between UCP activators and nonspecific uncouplers.

FIG. 7A shows the level of DiSC3 fluorescence for YUCP and YWT cells in the presence of various test and control agents.

FIG. 7B presents the data shown in FIG. 7A as a ratio of the fluorescence level in the presence of the test agent compared to the control level in the absence of the test agent.

FIG. 8 illustrates the results of triplicate fluorescence measurements on UCP1 and YWT cells for 3 hits identified from plate 9547.

FIG. 9 provides dose response curves for the UCP1 activator BrPalm, and for 2 additional UCP1 selective compounds (Compound C and Compound D). Solid lines show activity in UCP1 and dotted lines in YWT.

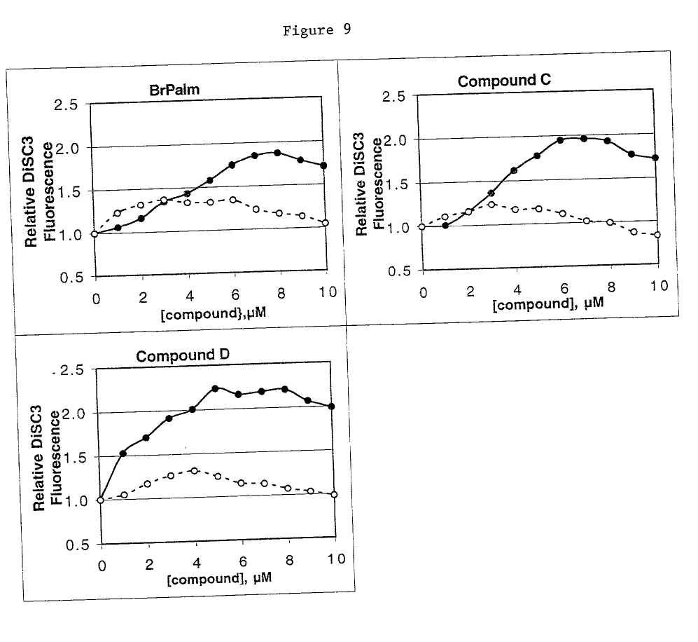

FIG. 10 provides the results of a 384-well screen of 320 test agents using control YWT cells, hybrid-hUCP2- and hybrid-hUCP3-expressing cells. The primary hit identified in the hUCP3 cells is specific to UCP3 because it increases the fluorescence in the hUCP3 cell only.

FIG. 11 provides an overall view of a screening strategy including secondary screening and analysis steps.

FIG. 12 illustrates the construction of hybrid forms of UCP.

FIG. 13 provides the DNA sequence for a hybrid hUCP2.

### DETAILED DESCRIPTION OF THE INVENTION AND PREFERRED EMBODIMENTS

I. Introduction

The present invention provides methods for the rapid and efficient identification of compounds with uncoupling activity in mitochondria. The methods can be used to detect modifiers of proteins with uncoupling activity, or can be used to identify molecules with intrinsic, i.e., uncoupling protein-independent, uncoupling activity. Such compounds can be activators or inhibitors of uncoupling activity and/or uncoupling proteins. This invention is based on the surprising discovery that, using particular dyes, it is possible to detect changes in mitochondrial membrane potential using high-throughput, homogeneous assay formats.

In general, the methods provided herein include several steps, including contacting a cell with a fluorescent probe whose fluorescence is a function of the mitochondrial membrane potential in the cell, contacting the cell with a test agent, and detecting an effect of the test agent on the fluorescence of the cell. In certain cases, the test agent will alter the mitochondrial membrane potential, thereby altering the fluorescence in the cell and allowing the identification of agents with the ability to modulate uncoupling.

In certain embodiments, the cell expresses an uncoupling protein, e.g., by recombinant means, and a secondary step is performed in which the ability of the test agent to modulate the fluorescence of a cell that does not express the uncoupling protein is assessed. In such embodiments, an ability of the test agent to modulate uncoupling activity in the presence of the uncoupling protein, but not in the absence of the uncoupling protein, indicates that the modulatory activity of the agent is specific for the uncoupling protein. Similar secondary steps can be performed in which the ability of a test agent to modulate uncoupling activity in a cell expressing one uncoupling protein is compared to the ability of the agent to modulate uncoupling activity in a second cell expressing another uncoupling protein. In such embodiments, an ability of the agent to modulate uncoupling activity in the presence of the first uncoupling protein, but not in the presence of the second uncoupling protein, indicates that the modulatory activity of the agent is specific for the first uncoupling protein. Alternatively, a compound that modulates uncoupling activity in the presence of any of a number of uncoupling proteins, but not in the absence of any uncoupling proteins, indicates that the agent is capable of modulating uncoupling activity in cells by modulating the activity of a plurality of uncoupling proteins.

The present methods are useful in the identification of compounds that are useful in the treatment of diseases or conditions associated with uncoupling activity. For example, compounds that are capable of modulating uncoupling activity, either intrinsically or by modulating uncoupling protein activity, are useful in the treatment of metabolic or weight disorders such as obesity. Compounds capable of modulating uncoupling are also useful in the study of the mechanisms, causes, and consequences of uncoupling in cells, for example by identifying proteins or other compounds that the compound associates with in vivo or in vitro, or by creating animal models of uncoupling related metabolic or weight disorders. Such animal models are useful, e.g., for the study of such disorders, as well as for the identification of compounds useful in the treatment or prevention of the disorders.

The present methods are also useful in the identification of compounds that modulate cellular processes associated with changes in mitochondrial membrane potential, such as apoptosis. In such embodiments, a cell is typically contacted with a test agent in the presence of an apoptosis-inducing compound or treatment, and the ability of the test agent to modulate the uncoupling associated with the compound or treatment is assessed. Test agents that are found to be capable of modulating apoptosis-associated uncoupling are useful in the study of apoptosis, as well as in the treatment of any of a large number of diseases and conditions associated with apoptosis, including, but not limited to, inflammatory diseases, viral infections, neurodegenerative diseases, cancers and heart disease, or as a method of inducing apoptosis in undesired cells in vivo.

Kits for practicing the present methods are also provided.

II. Definitions

A “test agent” refers to any molecule, material, or treatment that is tested in a screen. The molecule may be randomly selected for inclusion in the screen, or may be included because of an a priori expectation that the molecule will give a positive result in the screen. Molecules can include any known chemical or biochemical molecule, including peptides, nucleic acids, carbohydrates, lipids, or any other organic or inorganic molecule. A “test agent” can also refer to non-molecular entities, such as electromagnetic radiation or heat.

When a test agent is said to “modulate” the activity of an uncoupling protein, or of the uncoupling activity in a cell, this means that the uncoupling activity in the mitochondria of the cell is detectably altered. In certain embodiments, the uncoupling activity will be manifest as fluorescence, and a “modulation” can be detected as a difference in, e.g., fluorescence intensity. Preferably, such fluorescence will be measurable, and a “modulation” will comprise a statistically significant alteration in the fluorescence. However, a “modulation” can also refer to detection of a change by any means, such as a subjective determination by a human observer.

An “uncoupling protein” refers to any polypeptide that acts to alter the mitochondrial membrane potential in a cell, e.g., that dissipates the mitochondrial membrane potential. Uncoupling proteins include, but are not limited to, UCP1 (or “UCP;” see, e.g., Cassard et al., (1990) J Cell Biochem 43:255-64; see, also, GenBank Accession No. U28480); UCP2 (see, e.g., Fleury et al., (1997) Nature Genet. 15:269-272; see, also, GenBank Accession No. AF096289), UCP3 (see, e.g., Boss et al., (1997) FEBS Lett. 408:39-42; see, also, GenBank Accession No. NM 003356), UCP4 (see, e.g., Mao et al., (1999) FEBS Lett. 443:326-30; see, also, GenBank Accession No. AF110532), and BMCP1 (see, e.g., Sanchis et al., (1998) J. Biol. Chem. 273:34611-5; see, also, GenBank Accession No. AF078544), or any homolog, variant, fragment, or derivative thereof, from any source including humans. The ability of a polypeptide to alter mitochondrial membrane potential can be assessed using any method, including the herein-described homogeneous assays.

“Expressing” a protein in a cell means to ensure that the protein is present in the cell, e.g., for the purposes of a procedure of interest. In numerous embodiments, “expressing” a protein will comprise introducing a transgene into a cell comprising a polynucleotide encoding the protein, operably linked to a promoter, wherein the promoter is a constitutive promoter, or an inducible promoter where the conditions sufficient for induction are created. However, a cell that, e.g., naturally expresses a protein, can be used without manipulation and is considered as “expressing” the protein.

A “fluorescent probe” refers to any compound with the ability to emit light of a certain wavelength when activated by light of another wavelength.

“Fluorescence” refers to any detectable characteristic of a fluorescent signal, including intensity, spectrum, wavelength, intracellular distribution, etc.

“Membrane potential” refers to a difference in the electrical potential across a membrane such as a mitochondrial membrane. In the context of the present invention, such differences reflect transmembrane differences in the concentrations of charged molecules, such as sodium, potassium, and, particularly in the case of mitochondrial membranes, protons.

“Detecting” fluorescence refers to assessing the fluorescence of a cell using qualitative or quantitative methods. Preferably, the fluorescence is determined using quantitative means, e.g., measuring the fluorescence intensity, spectrum, or intracellular distribution, allowing the statistical comparison of values obtained from test agents and control values. The level can also be determined using qualitative methods, such as the visual analysis and comparison by a human of multiple samples, e.g., samples detected using a fluorescent microscope or other optical detector (e.g., image analysis system, etc.)

An “alteration” or “modulation” in fluorescence refers to any detectable difference in the intensity, intracellular distribution, spectrum, wavelength, or other aspect of fluorescence in the presence of a test agent or other compound. Preferably, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems described herein, or can reflect a subjective detection of an alteration by a human observer.

An assay performed in a “homogeneous format” means that the assay can be performed in a single container, with no manipulation or purification of any components being required to determine the result of the assay, e.g., a test agent can be added to an assay system and any effects directly measured. Often, such “homogeneous format” assays will comprise at least one component that is “quenched” or otherwise modified in the presence or absence of a test agent. In numerous embodiments of the present invention, for example, the fluorescent dyes are present within the mitochondrial matrix in the absence of uncoupling activity, and the fluorescence is quenched. In the presence of uncoupling activity, however, the dyes move to the extramitochondrial space, thereby reducing the level of quenching of the dye, and increasing the fluorescent signal in the cell.

A “secondary screening step” refers to a screening step whereby a test agent is assessed for a secondary property in order to determine the specificity or mode of action of a compound identified using the methods provided herein. For example, a compound found to modulate uncoupling activity in a cell expressing UCP1 can be assessed for an ability to modulate uncoupling activity in a cell that is not expressing UCP1, thereby determining the specificity of the modulatory activity. Such secondary screening steps can be performed on all of the test agents, or, e.g., on only those that are found to be positive in a primary screening step, and can be performed subsequently, simultaneously, or prior to a primary screening step.

“High-throughput screening” refers to a method of rapidly assessing a large number of test agents for a specific activity. Typically, the plurality of test agents will be assessed in parallel, for example by simultaneously assessing 96 or 384 agents using a 96-well or 384-well plate, 96-well or 384-well dispensers, and detection methods capable of detecting 96 or 384 samples simultaneously. Often, such methods will be automated, e.g., using robotics.

“Robotic high-throughput screening” refers to high-throughput screening that involves at least one robotic element, thereby eliminating a requirement for human manipulation in at least one step of the screening process. For example, a robotic arm can dispense a plurality of test agents to a multi-well plate.

A “multi-well plate” refers to any container, receptacle, or device that can hold a plurality of samples, e.g., for use in high-throughput screening. Typically, such “multi-well plates” will be part of an integrated and preferably automated system that enables the rapid and efficient screening or manipulation of a large number of samples. Such plates can include, e.g., 24, 48, 96, 384, or more wells, and are typically used in conjunction with a 24, 48, 96, 384, or more tip pipettors, samplers, detectors, etc.

A “permeabilizing agent” refers to any agent that acts to permeabilize the cell wall of yeast. Such agents may comprise enzymes that act to degrade the yeast cell wall, such as zymolyase or chitinase, or can comprise chemical agents that can permeabilize the yeast cell wall by chemical means.

“Zymolyase” refers to an enzyme that is capable of degrading the cell wall of yeast. Often, such enzymes are purified from Arthrobacter luteus, and comprise beta-1,3-glucan laminaripentaohydrolase activity, i.e., hydrolysis of glucose polymers linked by beta-1,3-bonds, producing laminaripentaose. Zymolyase is thought to comprise two enzymes, Zymolyase A (beta-1,3-glucan laminaripentaohydrolase) and Zymolyase B (alkaline protease).

III. Assays and Assay Components

A. Cells

Any of a number of cell types can be used in the present invention. For example, any eukaryotic cell, including plant, animal, and fungal cells can be used. In preferred embodiments, yeast cells will be used. As used herein, “cells” can include whole cells (untreated cells), permeabilized cells, isolated mitochondria, and proteoliposomes, e.g., proteoliposomes reconstituted with a UCP or another protein of interest. In particularly preferred embodiments, Saccharomyces cerevisiae, such as strains W303 or YWT, are used. The care and maintenance of cells, including yeast cells, is well known to those of skill in the art and can be found in any of a variety of sources, such as Freshney (1994) Culture of Animal Cells: A Manual of Basic Technique, Wiley-Liss, New York, Guthrie & Fink (1991), Guthrie and Fink, Guide to Yeast Genetics and Molecular Biology, Academic Press, Ausubel et al. (1999) Current Protocols in Molecular Biology, Greene Publishing Associates, and others.

Typically, when yeast cells are used, the cells will be permeabilized prior to the addition of the fluorescent dye and/or test agent. Such permeabilization steps rely on chemical agents, such as digitonin, SDS, DMF, chloroform, etc., or on enzymes such as zymolyase, lyticase, gluculase, chitinase, etc., that can permeabilize the yeast cell wall and that are well known to those of skill in the art. Such agents are described, e.g., in Guthrie & Fink (1991), and in Ausubel et al., both supra. In preferred embodiments, zymolyase is used. Zymolyase and other compounds are readily available from commercial sources, such as Promega, Zymo Research, SIGMA, Fluka, etc.

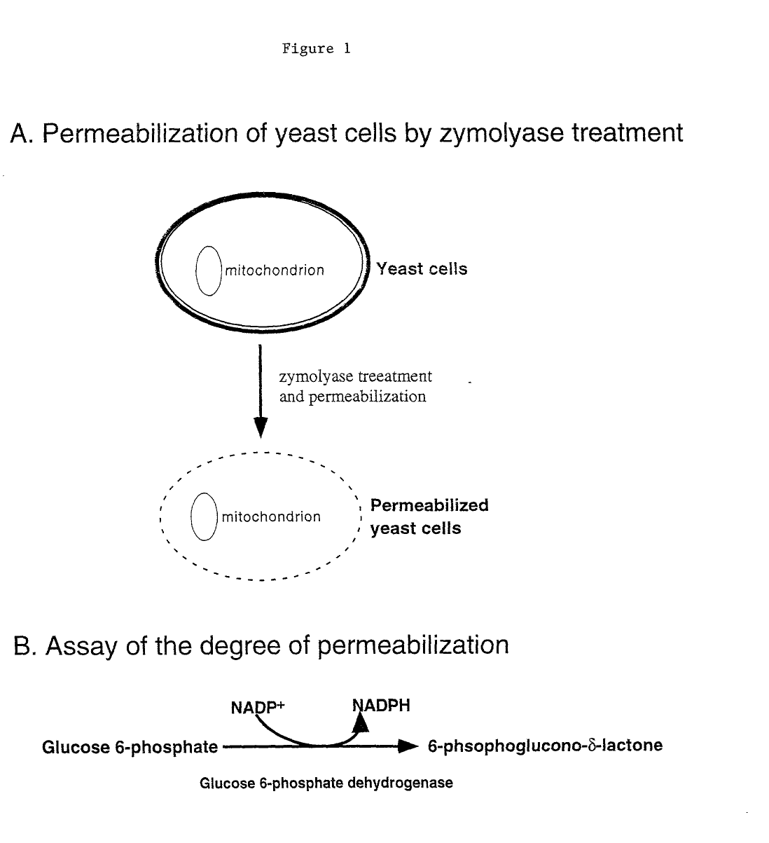

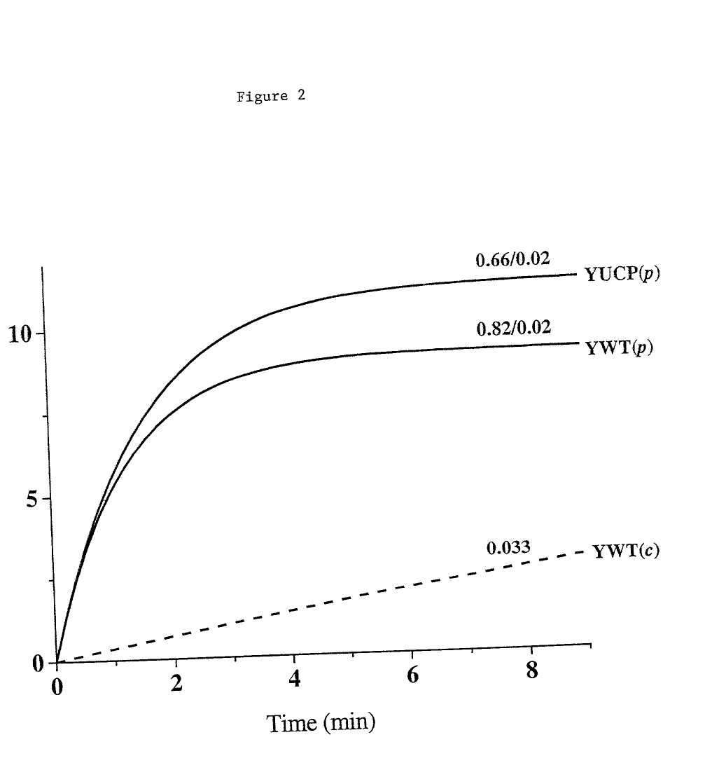

In preferred embodiments, the degree of permeabilization is assessed. For example, the enzyme glucose 6-phosphate dehydrogenase catalyzes the conversion of glucose 6-phosphate into δ-phosphoglucono-6-lactone. The rate of this reaction, under the experimental conditions provided herein, is limited by the availability of glucose 6-phosphate and NADP. Permeabilization of the yeast cells in the present assays increases the levels of these substrates, thereby increasing the rate of the reaction by, e.g., about 20-fold (see, e.g., FIGS. 1 and 2).

In other embodiments, mammalian, insect, or other metazoan cells can be used to test for agents that are capable of inducing apoptosis or that otherwise affect mitochondrial membrane potential. Any such cell type can be used, including primary cell lines, secondary cell lines, transformed cells, and others, and including whole (untreated) cells, permeabilized cells, isolated mitochondria, and proteoliposomes. For example, a number of cell types are described by the ATCC, or in Freshney (1994), supra, any of which can be used. For example, murine myelomas, n51, VERO, HeT, SF9, CV-1, CHO, and other cells can be used. In preferred embodiments, a cell, e.g., an animal cell, that normally expresses a UCP protein can be used. For example, a brown adipose cell expressing UCP1 can be used, or a brain, muscle, or fat cell expressing UCP2 can be used.

Cells can be used at any of a wide range of densities, depending on the dye, the test agent, and the particular assay conditions. Preferably, a density of about OD₆₀₀=0.01 to 1 is used, more preferably between about 0.05 and 0.5, most preferably about 0.1.

B. Uncoupling Proteins

A large number of uncoupling proteins have been identified from numerous organisms, any of which can be used in the present invention. For example, UCP1, UCP2 (see, e.g. Fleury, et al. (1997) Nature Genetics 15:269; U.S. patent application Ser. No. 09/124,293, filed Jul. 29, 1998), UCP3, UCP4 (see, e.g., Mao et al. (1999) FEBS Lett., 443:326), BMCP1 (see, e.g., Sanchis, et al. (1998) J. Biol. Chem. 273:34611) or homologs or derivatives thereof, can be used. UCPs have been shown to possess proton transporting activity, and to typically have six alpha-helical transmembrane domains. UCP1-4 are homologous to each other. UCP proteins suitable for the present invention can be derived from, e.g., mammals, plants, fish, worms, insects, fungi, or any other eukaryote. In numerous embodiments, a hamster UCP sequence is used. Amino acid and nucleotide sequences for a multitude of UCP proteins can be found, e.g., by accessing GenBank at the National Institute of Biotechnology Information (www.ncbi.nlm.nih.gov) (see, e.g. accession numbers Y18291, NM_{—}003356.1, AF096289, AF110532, AF036757, AF092048, and others). UCP proteins are also described, e.g., in Tartaglia (1988), U.S. Pat. No. 5,853,975, and in Science 280:1369 (1998).

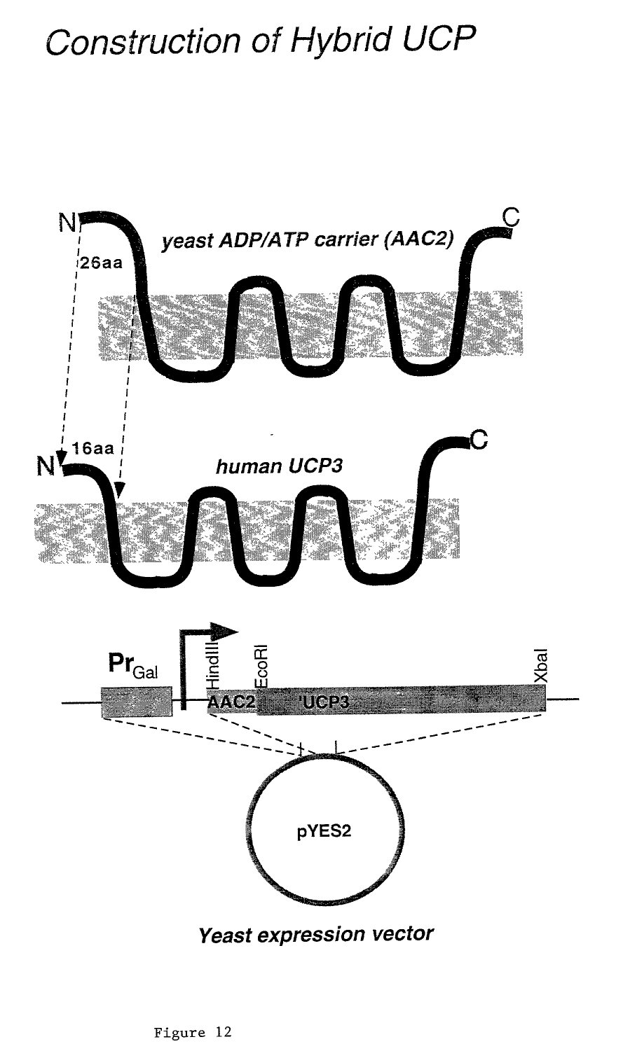

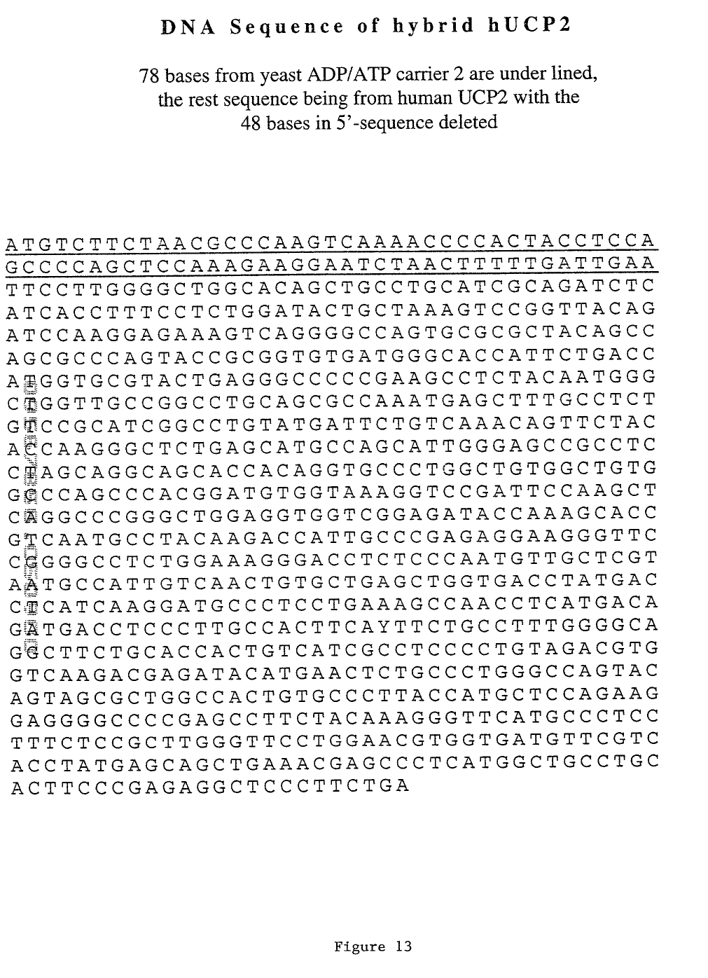

In preferred embodiments, a hybrid form of an uncoupling protein will be used. Such hybrid forms can include a UCP protein, or fragment thereof, as well as a heterologous polypeptide sequence such as a label, antigenic sequence, or, preferably, a leader sequence that facilitates the insertion of the protein into the mitochondrial membrane. For example, hybrid forms of UCPs that include a leader sequence from the yeast AAC2 protein (see, e.g., FIGS. 12 and 13) can be used.

C. Expressing Uncoupling Proteins in Cells

In numerous embodiments, one or more UCP proteins will be expressed in yeast or other cells. Methods for expressing heterologous proteins in yeast and other cells are well known to those of skill in the art, and are described, e.g., in Ausubel (1999), Guthrie and Fink (1991), Sherman, et al. (1982) Methods in Yeast Genetics, Cold Spring Harbor Laboratories, Freshney, and others. Typically, in such embodiments, a polynucleotide encoding a UCP protein will be operably linked to an appropriate expression control sequence for the particular host cell in which the UCP protein is to be expressed. Any of a large number of yeast promoters can be used, including inducible promoters such as GAL1 (Johnson and Davies (1984) Mol. Cell Biol. 4:1440), ADH2 (Russell, et al. (1983) J. Biol. Chem. 258:2674-2682, PHO5 (EMBO J. 6:675-680 ((1982)), MFα1 (Herskowitz and Oshima (1982), in The Molecular Biology of the Yeast Saccharomyces (Strathem, et al., eds.), Cold Spring Harbor Labs., N.Y), and others. Additional elements such as polyadenylation signals, 5′ and 3′ untranslated sequences, etc. are also described in such references.

In metazoan cells, promoters and other elements for expressing heterologous proteins are commonly used and are well known to those of skill. See, e.g., Cruz & Patterson (1973) Tissue Culture, Academic Press; Meth. Enzymology 68 (1979), Academic Press; Freshney, 3^{rd} Edition (1994) Culture of Animal Cells: A Manual of Basic Techniques, Wiley-Liss. Promoters and control sequences for such cells include, e.g., the commonly used early and late promoters from Simian Virus 40 (SV40), or other viral promoters such as those from polyoma, adenovirus 2, bovine papilloma virus, or avian sarcoma viruses, herpes virus family (e.g., cytomegalovirus, herpes simplex virus, or Epstein-Barr Virus), or immunoglobulin promoters and heat shock promoters (see, e.g. Sambrook, Ausubel, Meth. Enzymology (1979, 1983, 1987), Pouwells, et al., supra (1987)). In addition, regulated promoters, such as metallothionein, (i.e., MT-1 and MT-2), glucocorticoid, or antibiotic gene “switches” can be used. Enhancer regions of such promoters can also be used.

Expression cassettes are typically introduced into a vector that facilitates entry of the expression cassette into a host cell and maintenance of the expression cassette in the host cell. Such vectors are commonly used and are well know to those of skill in the art. Numerous such vectors are commercially available, e.g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e.g., LEU2, URA3, TRP1, HIS3, GFP), centromeric sequences, etc. Examples of such vectors include Yeast Integrating Plasmids (e.g., YIp5), Yeast Replicating Plasmids (e.g.,YRp series plasmids), and pGPD-2. Also suitable are yeast expression plasmids such as YEp6, YEp13, YEp4, etc. Such plasmids are described, e.g., in Botstein, et al., (1979) Gene 8:17-24, Broach, et al. (1979) Gene 8:121-133, Parents (1985), YEAST, and others.

For expression in mammalian cells, any of a number of vectors can be used, such as pSV2, pBC12BI, and p91023, as well as lytic virus vectors (e.g., vaccinia virus, adenovirus, baculovirus), episomal virus vectors (e.g., bovine papillomavirus), and retroviral vectors (e.g., murine retroviruses).

D. Fluorescent Probes

The present invention can be practiced using any dye whose distribution, intensity, or spectral characteristics change with changing mitochondrial membrane potential. For example, any probe listed in the chapter on potentiometric probes in the Molecular Probes (Eugene, Ore.) catalog can be used. Such probes include fast response probes (including styryl and hybrid oxonol probes) and, preferably, slow response probes, including, but not limited to, Carbocyanine probes (e.g., DiOC₂(3), DiOC₂(5), DiOC₅(3), DiOC₇(3), DiSC₂(5), DiSC₃(5), DiIC₁(3), and JC-1), Rhodamine probes (e.g., tetramethylrhodamine methyl and ethyl esters, Rhodamine 123), Oxonol probes (e.g., Oxonol V, Oxonol VI, DiBAC₄(3), DiBAC₄(5), and DiSBAC₂(3)), and Merocyanine probes (e.g., merocyanine 540). In addition, O-safranine can be used to monitor mitochondrial membrane potential by measuring the absorption of visible light (OD530 nm-OD490 nm). The suitability of any of the herein-described probes in the present assays can readily be assessed, e.g., by contacting one or more cells, mitochondria, etc. in a homogeneous format with the dye and with a compound that is known to alter the mitochondrial membrane potential (e.g., FCCP or CCCP), and detecting the fluorescence in the sample. Any dye that allows the detection of a change in fluorescence under such conditions can be used in the present assays.

In preferred embodiments, DiSC₃(5), a lipophilic cation that distributes itself according to membrane potential, or derivatives or analogs thereof, is used. DiSC₃(5), also known as 3,3′-dipropylthiadicarbocyanine iodide, or DiSC3, is a carbocyanine dye of molecular weight 546.53 and is typically supplied in a solid form. To prepare a DiSC3 solution, it can be dissolved in DMSO. DiSC3 has an absorptive frequency of 651 nm, and an excitation frequency of 675 nm. DiSC3 itself, as well as additional information about DiSC3, can be obtained from Molecular Probes (Oregon); see, e.g. www.probes.com. See, also, Bunting, et al. Biophys. J. 56:979 (1989).

Such dyes can be added at any concentration that allows detection using standard methodology. Preferably, a concentration of between about 0.01 μM and about 1 μM is used, more preferably between about 0.05 μM and about 0.5 μM and, most preferably, about 0.1 μM. Typically, such dyes will be added to cells for between about 5 and about 30 minutes, and will be added using a buffer. A preferred buffer suitable for use in the methods of the present invention includes 290 mM mannitol, 20 mM potassium phosphate, 0.5 mM EGTA, 0.2 mg/ml bovine serum albumin, 2 μg/ml oligomycin, and 5 mM glycerate α-phosphate.

E. Test Agents

Essentially any chemical compound can be used as a potential activity modulator in the assays of the invention, although most often compounds that can be dissolved in aqueous or organic (especially DMSO-based) solutions are used. The assays are designed to screen large chemical libraries by automating the assay steps and providing compounds from any convenient source to assay, which are typically run in parallel (e.g., in microtiter formats on microtiter plates in robotic assays). It will be appreciated by those of skill in the art that there are many commercial suppliers of chemical compounds, including Sigma Chemical Co. (St. Louis, Mo.), Aldrich Chemical Co. (St. Louis, Mo.), Sigma-Aldrich (St. Louis, Mo.), Fluka Chemika-Biochemica Analytika (Buchs Switzerland), and the like.

In one preferred embodiment, high-throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (e.g., amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton, et al., Nature, 354:84-88 (1991)). Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Russia, Tripos, Inc., St. Louis, Mo., ChemStar, Ltd., Moscow, RU, 3D Pharmaceuticals, Exton, Pa., Martek Biosciences, Columbia, Md., etc.).

F. High-Throughput Format

As noted, the invention provides in vitro assays for uncoupling activity in a high-throughput format. Control reactions that measure uncoupling activity in a reaction that does not include an uncoupling activity modulator are optional, as the assays are highly uniform. However, such optional control reactions are appropriate and increase the reliability of the assay. Accordingly, in a preferred embodiment, the methods of the invention include such a control reaction.

In some assays, it will be desirable to have positive controls to ensure that the components of the assays are working properly. For example, a known activator of uncoupling activity can be incubated with one sample of the assay, and the resulting increase in uncoupling activity determined according to the methods herein. In preferred embodiments, CCCP, or carbonyl-cyanide p-chlorophenylhydrazone, is used. CCCP can be added at any concentration sufficient to effect a detectable amount of uncoupling. For example, 0.1 μM, 1 μM, 10 μM, 100 μM or 1 mM can be used. In preferred embodiments, 0.1 to 5 μM can be used and, more preferably, 2 μM is used. CCCP is readily available from commercial sources, e.g., Sigma. See, e.g., Heytler, et al. (1962) Biochem .Biophys. Res. Commun., 7:272.

In embodiments wherein an uncoupling protein is expressed in a cell, a known modulator of the uncoupling protein is preferably used. For example, the commercially available UCP1 activator 2-bromo-palmitate (BrPalm) can be used (at, e.g., 5 to 10 μM), thereby providing a UCP1-specific increase in uncoupling activity. In addition, a known inhibitor of UCP activity can be added, and the resulting decrease in uncoupling activity similarly detected. For example, GDP can be used to inhibit UCP1, at, e.g., 100 μM.

In the high-throughput assays of the invention, it is possible to screen up to several thousand different modulators in a single day. In particular, each well of a microtiter plate can be used to run a separate assay against a selected potential modulator, or if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000-1,000,000 different compounds are possible using the integrated systems of the invention.

II. Compositions, Kits and Integrated Systems

The present invention provides compositions, kits and integrated systems for practicing the assays described herein. For example, an assay composition having a source of cells (in certain embodiments expressing a UCP or other uncoupling protein), a fluorescent dye whose fluorescence reflects the membrane potential of the mitochondria in the cell, and one or more compounds that can be used as positive or negative controls, e.g., FCCP, CCCP, GDP and/or BrPalm, is provided by the present invention. Additional assay components as described above are also provided. For instance, a solid support or substrate in which the assays can be carried out can also be included. Such solid supports include membranes (e.g., nitrocellulose or nylon), a microtiter dish (e.g., PVC, polypropylene, or polystyrene), a test tube (glass or plastic), a dipstick (e.g., glass, PVC, polypropylene, polystyrene, latex, and the like), a microcentrifuge tube, or a glass, silica, plastic, metallic or polymer bead or other substrate such as paper. Most commonly, the assay will use 96, 384 or 1536 well microtiter plates.

The invention also provides kits for practicing the uncoupling screening assays described above. The kits can include any of the compositions noted above, and optionally further include additional components such as instructions to practice a high-throughput method of screening for an uncoupling activity modulator, one or more containers or compartments (e.g., to hold the cells, test agents, controls, dyes, or the like), a control activity modulator, a robotic armature for mixing kit components, and the like.

The invention also provides integrated systems for high-throughput screening of potential modulators of uncoupling activity. Such systems typically include a robotic armature which transfers fluid from a source to a destination, a controller which controls the robotic armature, a label detector, a data storage unit which records label detection, and an assay component such as a microtiter dish.

A number of well-known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art.

Any of the assays for compounds that modulate uncoupling activity, as described herein, are amenable to high-throughput screening. High-throughput screening systems are commercially available (see, e.g., Zymark Corp. (Hopkinton, Mass.); Air Technical Industries (Mentor, Ohio); Beckman Instruments, Inc. (Fullerton, Calif.); Precision Systems, Inc., (Natick, Mass.), etc.). Such systems typically automate entire procedures including all sample and reagent pipetting, liquid dispensing, timed incubations, and final readings of the microplate in detector(s) appropriate for the assay. These configurable systems provide high-throughput and rapid start up as well as a high degree of flexibility and customization. The manufacturers of such systems provide detailed protocols for the various high-throughput systems.

Optical images viewed (and, optionally, recorded) by a camera or other recording device (e.g., a photodiode and data storage device) are optionally further processed in any of the embodiments described herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS™, OS2™ WINDOWS™, WINDOWS NT™ or WINDOWS95™ based machines), MACINTOSH™, or UNIX based (e.g., SUN™ work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

III. Identification of Apoptosis-Modulating Compounds

It will be appreciated that the methods provided herein can be used to screen any test agent for its ability to alter mitochondrial membrane potential in a cell. Thus, in addition to molecules with intrinsic uncoupling activity, or that modulate uncoupling proteins, these methods can be used to identify agents that indirectly affect mitochondrial membrane potential. For example, molecules that induce apoptosis, which is characterized by a loss of mitochondrial membrane potential, can be screened. In a typical embodiment, cells are exposed to a ΔΨm-dependent fluorescent probe, contacted with a test agent, and the fluorescence is detected. Preferably, such methods are performed in a high-throughput format, allowing the rapid and efficient screening of a large number of test agents. Test agents identified as “hits” in such screens can be subject to a secondary screening step, for example, by screening for other apoptosis markers, such as annexin V, propidium iodide, etc.

V. Secondary Screening Steps

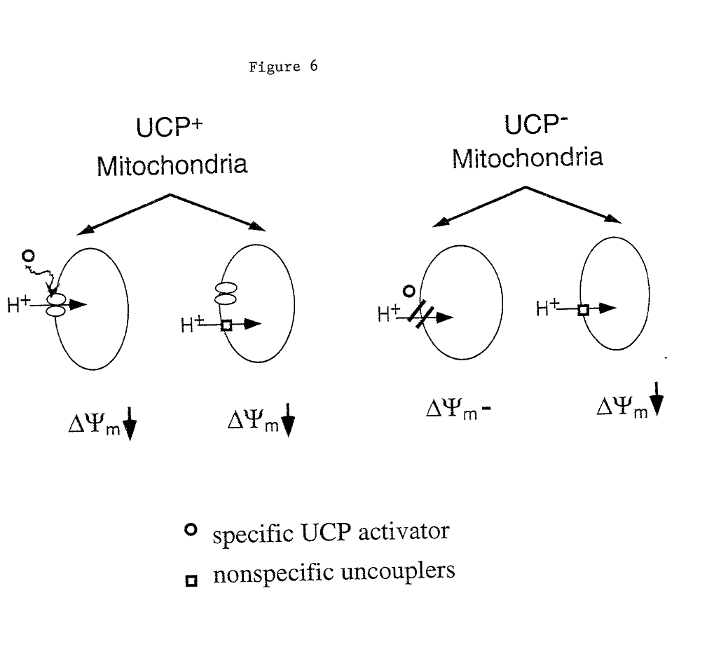

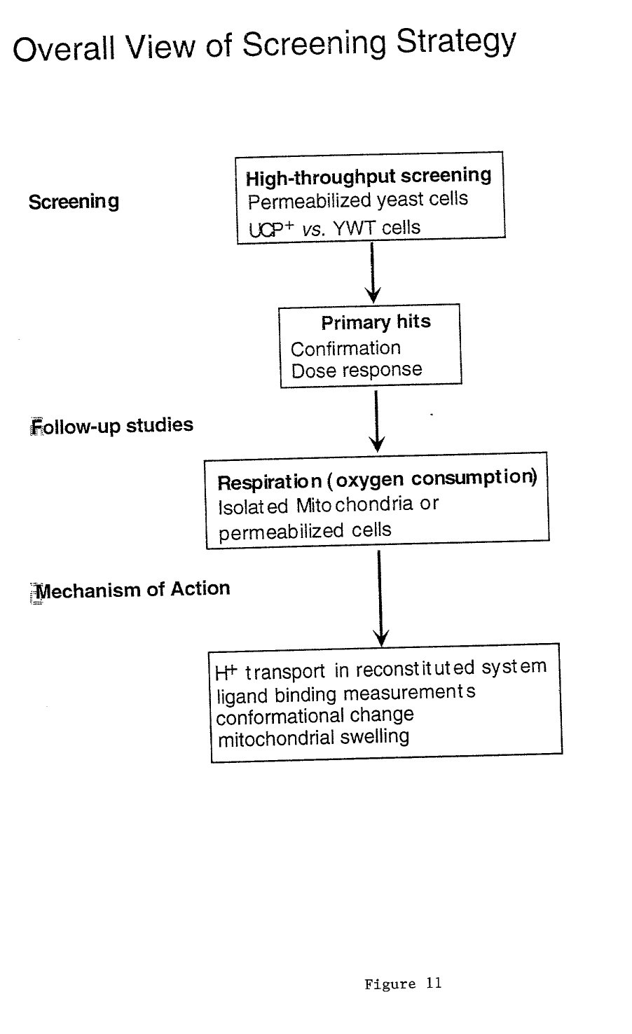

Compounds identified using the present methods can be further screened or analyzed to better assess their role in uncoupling or in modulating the activity of one or more uncoupling proteins (see, e.g., FIGS. 6 and 11).

In preferred embodiments, compounds screened against a cell expressing one or more uncoupling proteins will also be screened against cells that do not express the protein. In this way, the specificity of the compound for the uncoupling protein is assessed. Compounds that are found to cause an increase in fluorescence in both YUCP (expressing) and YWT (non-expressing) cells are either chemical uncouplers (i.e., cause uncoupling in an UCP-independent manner) or are themselves fluorescent under the experimental excitation and emission wavelengths. These possibilities can be easily distinguished by examining the fluorescence of the compound alone, i.e., isolated from cells or mitochondria. If the compound does not fluoresce on its own, it is very likely that the compound is a chemical uncoupler. Chemical uncouplers can be readily detected by, e.g., measuring mitochondrial membrane potential or oxygen consumption.

In certain embodiments, compounds screened against a particular uncoupling protein are subsequently screened against another uncoupling protein. For example, compounds found to modulate uncoupling activity in cells expressing UCP1 are then analyzed for the ability to modulate uncoupling activity in cells expressing UCP2. In preferred embodiments, multiple types of cells will be screened simultaneously.

In certain embodiments, compounds will be screened that affect multiple uncoupling proteins. For example, it may be desirable to find a compound that modulates both UCP3 and UCP4. In typical such embodiments, cells expressing UCP3 or UCP4 are screened simultaneously and, based on the fluorescence levels, UCP3-specific and UCP4-specific hits are identified. Compounds that modulate both UCP3 and UCP4 can then be distinguished from unspecific hits by screening the compounds against cells that do not express any UCP, or cells that only express a UCP other than UCP3 or UCP4. Compounds that specifically modulate UCP3 and UCP4 will be detected in UCP3 and UCP4 expressing cells, but not in the control cells that do not express UCP3 or UCP4.

In preferred embodiments, compounds identified using the assays described above will be tested using independent assays for an effect on mitochondrial respiration. For example, the primary hits derived from the membrane potential screens will be tested for their ability to alter mitochondrial oxygen consumption. A UCP specific activator should enhance the oxygen consumption rate, whereas a specific inhibitor should decrease the rate. Oxygen consumption can be conveniently measured using a Clark-type oxygen electrode, either in permeabilized cells or in isolated mitochondria oxidizing appropriate substrates. Cells can be fresh cells isolated from an appropriate tissue or mammalian cell cultures over-expressing the UCP. Permeabilization can be easily achieved by inclusion of 25 μg/mL digitonin. Substrates can be malate+glutamate or succinate+rotenone. The fold of increase or decrease in oxygen consumption of a hit compound will be measured relative to the basal rate in the absence of the compound as an index of the potency of the compound.

A. Identifying the Mechanism of Action

When the primary hits are confirmed using the oxygen consumption studies, further tests will be employed to determine the mechanism of action. For example, H⁺ transport activity in a reconstituted system can be assessed. In such embodiments, a UCP is isolated from a mitochondria and reconstituted into a phospholipid vesicle. When a potassium gradient is imposed across the membrane (i.e., by addition of the K⁺ ionophore valinomycin), H⁺ is driven in the opposite direction of the K⁺ gradient. The H⁺ transport activity can then be measured with either a fast-response pH electrode or a suitable fluorescent dye that is pH sensitive (e.g., pyranine). The increase solicited by the hit compound will be measured as an index of its potency. Such analyses will establish whether the hit compound is indeed interacting with the UCP.

In addition, the ability of a primary hit to bind to isolated UCP or mitochondria can be assessed. To study how the compound interacts with the UCP or mitochondria, both the kinetics and equilibrium binding can be measured using standard methods. The hit compound can be labeled, e.g., radio-labeled or labeled with a fluorescent dye. The binding constants (K_{D}) and rate constants can be measured using standard methods and compared to determine the best drug candidate.

Another method to further study primary hits is to assess any conformational change induced by the hit compound on a UCP protein. It is likely that hit compounds bind to the UCP and induce a conformational change in the UCP, such that the H⁺ ntransport activity of this protein is enhanced or decreased. Alternatively, the compound may not induce a conformational change but instead may provide necessary groups essential for H⁺ transport activity (such as free fatty acids provide the carboxyl groups for UCP1). The conformational change can be measured by proteolytic reaction—i.e., the hit compound may facilitate or retard the digestion, in either case indicating a conformational change induced by the ligand.

B. Animal Studies

Compounds identified using the present methods can readily be administered into a laboratory animal such as mouse or rat. The modulation of uncoupling activity in the animal can be assessed using any of large number of methods, including by measuring food intake, weight change, fat content, blood glucose level, or free fatty acid levels in the animal. In addition, basal energy expenditure can be monitored by measuring oxygen consumption, heat production, etc.

The invention will be described in greater detail by way of specific examples. The following examples are offered for illustrative purposes, and are not intended to limit the invention in any manner. Those of skill in the art will readily recognize a variety of noncritical parameters that can be changed or modified to yield essentially the same results.

### IV. EXAMPLES

A. Permeabilization of Yeast Cells

To make the cells permeable to externally added compounds, the yeast cells were treated with zymolyase. The spheroplasts were cultured in full media for 0.5 hours and pelleted. The pellets were resuspended in a lysis buffer and stored at about −80° C. The permeabilized cells can be kept in this way for at least one half year without loss of activity.

The degree of permeabilization was monitored by the glucose 6-phosphate dehydrogenase reaction (see, FIG. 2). Under the experimental conditions, this reaction is rate-limited by the availability of the substrates NADP and glucose 6-phosphate. The rate of the reaction in the permeabilized cells was typically 20-fold faster than in the untreated cells, indicating that most of the hydrophilic compounds must have penetrated the cell membrane and reached the mitochondria.

B. Detecting Alterations in Fluorescence Using Known Modulators of UCP1

To assess the effects of known modulators of uncoupling activity and of uncoupling protein activity, the following fluorescence intensity measurements were carried out: Permeabilized cells were combined with the fluorescent dye DiSC3 in buffer and incubated for about 5-60 min., usually less than about 30 min. The resulting fluorescence intensity (λ_{exc}=620 nm, λₑₘ=670 nm) corresponded to the basal ΔΨm. The mitochondria accumulate the fluorescent dye into the matrix in proportion to the ΔΨm, and at basal ΔΨm, the DiSC3 fluorescence is substantially quenched.

Upon addition of the UCP1 activator BrPalm (at 5 to 10 μM), the mitochondria became more uncoupled due to the activation of UCP1. As a result, the ΔΨm dropped rapidly, allowing the dye to redistribute to the extramitochondrial space, resulting in a higher fluorescence level. Because GDP is a specific inhibitor of UCP1, it was expected to recouple the mitochondria in the presence of UCP1 and BrPalm. When 100 μM of GDP was added in the presence of BrPalm, the fluorescence reverted back to the level close to basal intensity.

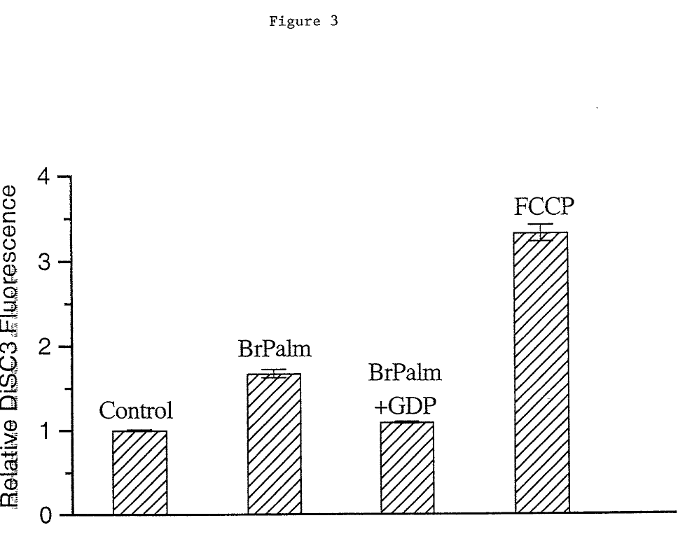

CCCP, or carbonyl-cyanide p-chlorophenylhydrazone, chemically uncouples the mitochondria. When even a low amount of CCCP (1 μM) was added to the cells, fluorescence reaches a maximum level. See, e.g., FIG. 3, providing results from an experiment using BrPalm, GDP, and the uncoupling compound FCCP.

C. The Plasma Membrane Does Not Contribute to the Fluorescence

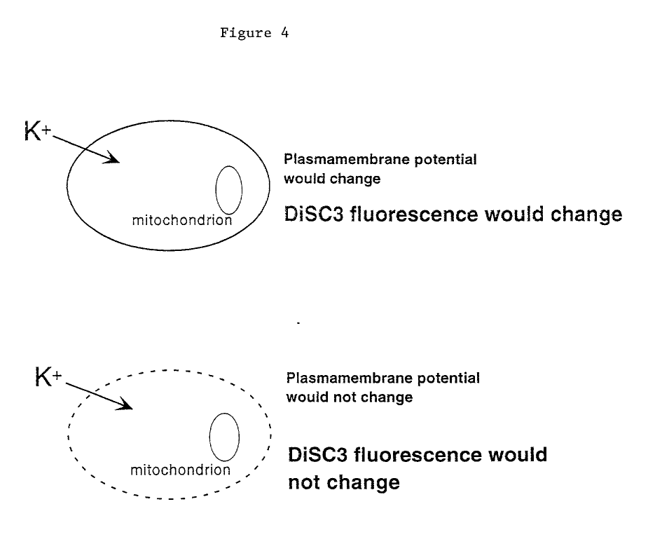

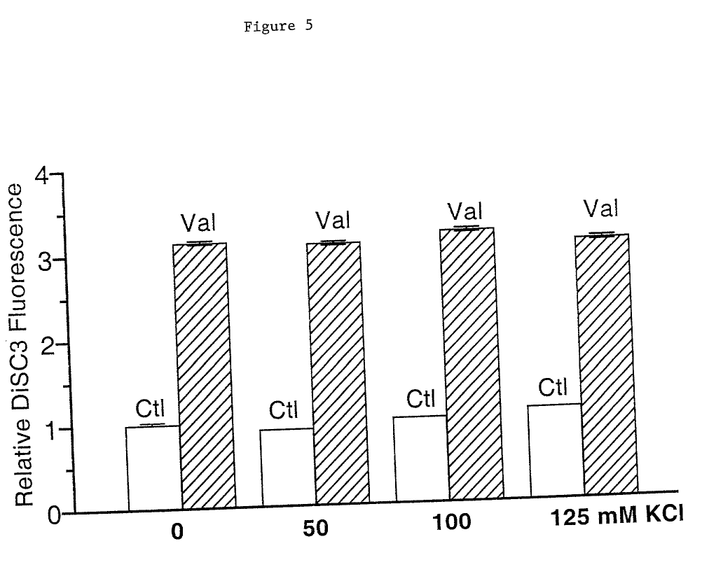

To test whether the fluorescence intensity also reflects the plasma membrane potential, the permeabilized cells were incubated with increasing concentrations of KCl. Because K⁺ changes the plasma membrane potential, but not the mitochondrial membrane potential (ΔΨm), any changes in the fluorescence intensity resulting from the KCl would indicate that the intensity also reflects the plasma membrane potential (see, e.g.,, FIG. 4). When 0 to 125 mM KCl was added to the cells, no change in fluorescence intensity was observed, indicating that the DiSC3 fluorescence essentially reflects the ΔΨm (see, e.g., FIG. 5).

D. Adapting to High-Throughput Screening in 96-Well Plates

A Beckman Multimek™ 96 Automated 96-channel Pipettor was programmed to mix test compounds from master plates with the buffer containing freshly premixed permeabilized cells (0.1 OD) and DiSC3 (0.1 μM) and transfer this mixture to black 96-well plates. An LJL analyst was used in the fluorescence intensity mode to measure the DiSC3 fluorescence. Pilot experiments showed that the fluorescence intensity is stable up to 40 min. A total of about 3,300 compounds were screened in this way to test the feasibility of this method.

From a total of 3,300 compounds, 77 activator-type and 20 inhibitor-type compounds were identified while screening against the UCP-expressing cells YUCP. The hits were picked up and screened again against YUCP. In this case, about 80% of the primary hits were confirmed.

To identify UCP-specific hits, the positive hits were screened against the control (YWT) cells, which do not express UCP (UCP1). A UCP-specific hit should not elicit any change in the fluorescence intensity in the control cells. When the above compounds were screened against the control cells expressing no UCP (YWT), 7 showed up as UCP1 specific activators because they did not elicit an increase in the fluorescence in the YWT cells, and 2 compounds behaved as specific inhibitors.

TABLE 1
3,300 compounds were screened against UCP1-expressing cells.

| | Hits | Confirmed | UCP1-specific |
|---|---|---|---|
| Activators | 77 | 66 | 7 activators (0.2%) |
| Inhibitors | 20 | 9 | 2 inhibitors (0.06%) |

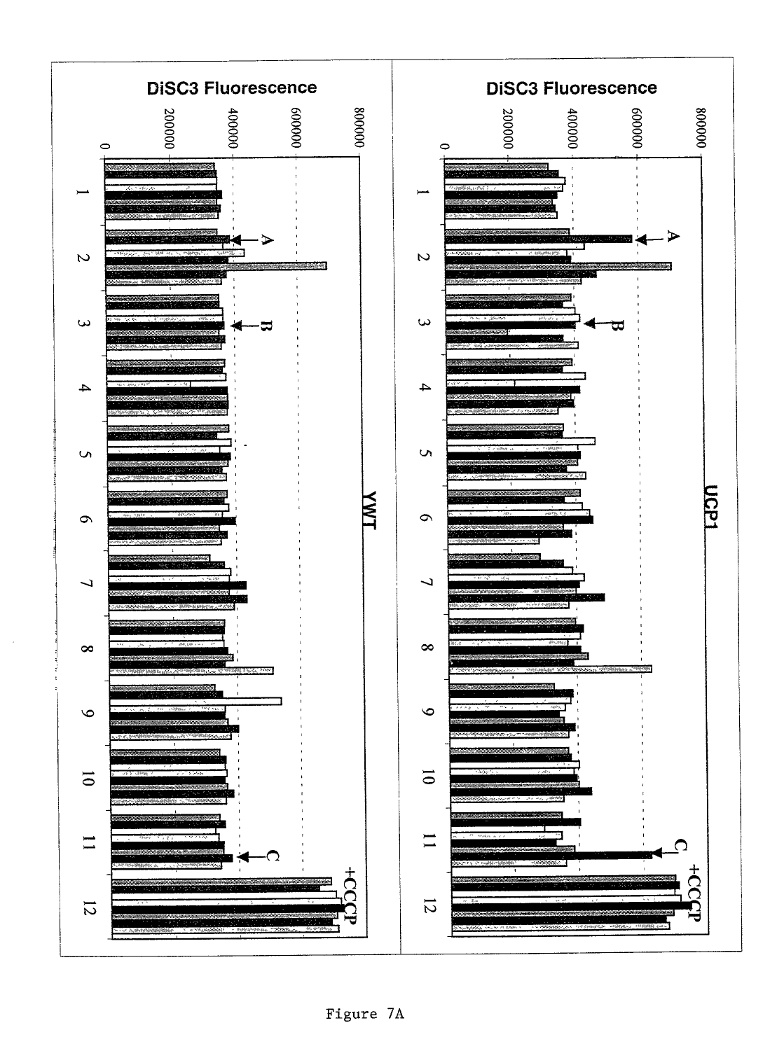

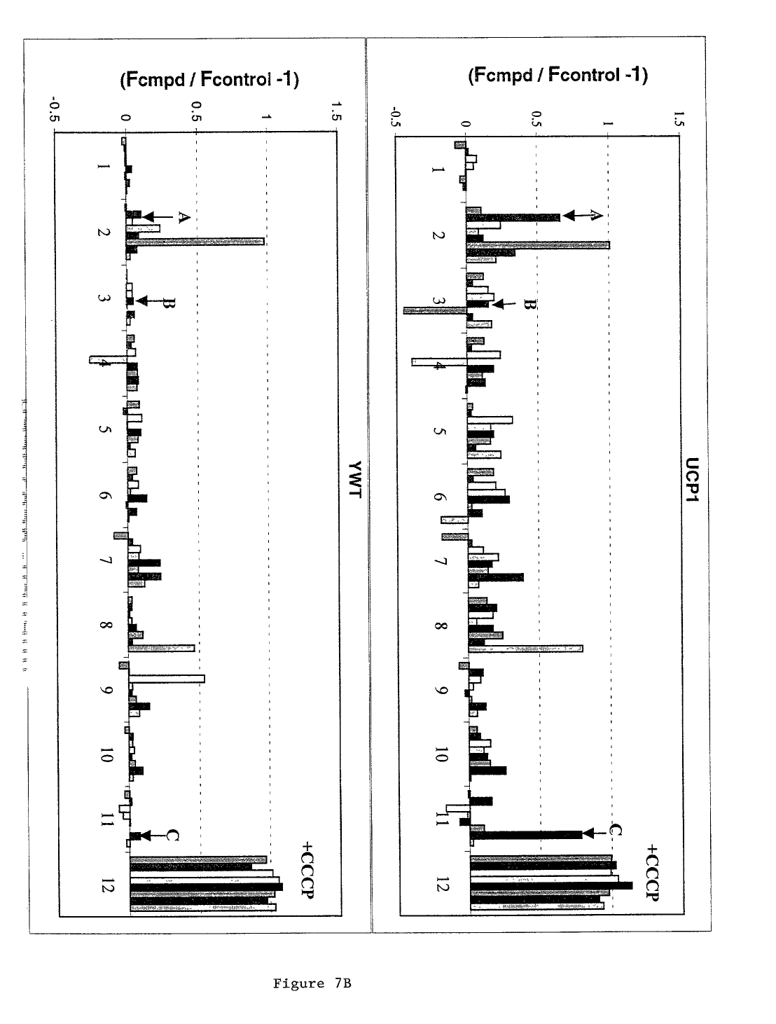

Data are presented in FIG. 7B after transformation of the fluorescence data according to (F_{compd}/F_{control}−1), where F_{compd} is the fluorescence intensity of a given well (as shown in FIG. 7A), and F_{control} is the basal fluorescence obtained as an average intensity in column 1 where no compound was added (instead an equivalent volume of dimethyl sulfoxide (DMSO) was added).

In this random plate (number 9547), there are two potentially specific positive hits, one in well B2 (arrow A), the other in well G11 (arrow C), and one potentially negative hit in well F3 (arrow B). The other hits are unspecific, because they hit both cells that express UCP1 and cells that do not express any UCP (YWT).

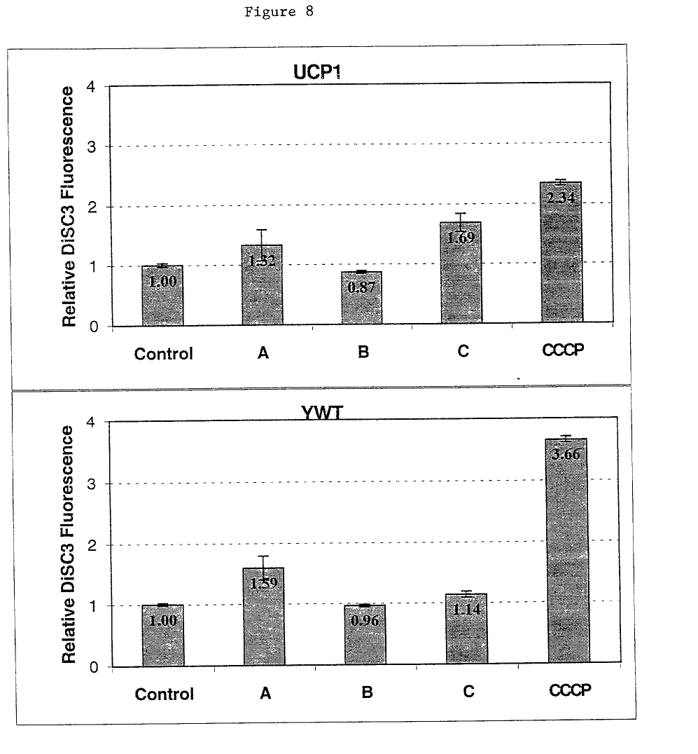

The 3 hits were picked and the same experiments were run in triplicate as shown below (see, e.g., FIG. 8). The hit 9547B2 (sample A) increased the fluorescence by 32% in UCP1 cells versus 59% in YWT cells, thus it is a false positive. Hit 9547F3 (sample B) decreased the fluorescence by 13% in YUCP cells compared to a decrease of 4% in YWT cells. It is therefore a potential inhibitor of UCP1. Most interestingly, hit 9547G11 (sample C) increased the fluorescence by 69% in YUCP cells in comparison to only 14% in YWT cells. Therefore, compound 9547G11 is a strong candidate for a specific UCP1-activator. Thus, in this example, two potential hits, i.e., one inhibitor and one activator, were found using the methods of the present invention.

E. Identification of a UCP3-Specific Primary Hit

320 test agents were screened in a 384-well format using control YWT cells, hUCP2- and hUCP3-expressing cells. The hUCP2 and hUCP3 used in this experiment represented hybrid forms of the proteins that included a leader sequence from yeast AAC2 (see, e.g., Hashimoto et al., (1999) Biochim. Biophys. Acta 1409:113-24) in order to facilitate insertion of the proteins into the yeast mitochondria. The construction of the hybrid hUCP2/hUCP3 are shown in FIG. 12, and the DNA sequence of the hybrid hUCP2 is shown in FIG. 13.

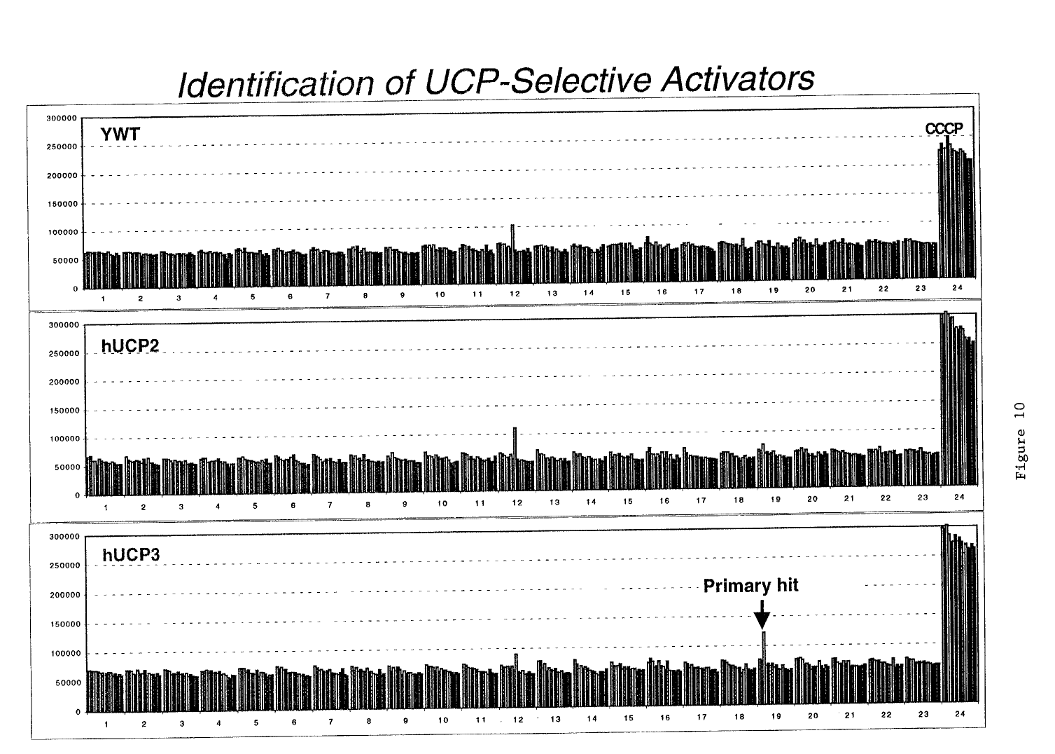

The results of this experiment are shown in FIG. 10. The primary hit identified in the hUCP3 cells is specific to hUCP3 because it increases the fluorescence in the hUCP3 cells only.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications and patent documents cited in this application are incorporated by reference in their entirety for all purposes to the same extent as if each individual publication or patent document were so individually denoted.

What is claimed is:

1. A method of screening a test agent for an ability to modulate the activity of an uncoupling protein, said method comprising:
(i) expressing said uncoupling protein in a cell;
(ii) introducing a fluorescent probe into said cell, wherein the fluorescence of said cell in the presence of said fluorescent probe is a function of the membrane potential in the mitochondria in said cell;
(iii) contacting said cell with said test agent; and
(iv) detecting the fluorescence in said cell;
wherein an alteration in the fluorescence in said cell in the presence of said test agent compared to the fluorescence in the absence of said test agent indicates an ability of said test agent to modulate said activity of said uncoupling protein; and
wherein said screening is performed in a homogeneous format.

2. The method of claim 1, further comprising a secondary screening step, wherein the fluorescence of the cell in the presence of said uncoupling protein is compared to the fluorescence of the cell in the absence of said uncoupling protein; and
wherein an ability of said test agent to modulate the fluorescence of the cell in the presence of said uncoupling protein, but not in the absence of said uncoupling protein, indicates that the activity of said test agent is specific to said uncoupling protein.

3. The method of claim 1, wherein said uncoupling protein comprises UCP1.

4. The method of claim 1, wherein said uncoupling protein comprises UCP2.

5. The method of claim 1, wherein said uncoupling protein comprises UCP3.

6. The method of claim 1, wherein said uncoupling protein comprises UCP4.

7. The method of claim 1, wherein said uncoupling protein comprises BMCP1.

8. The method of claim 1, wherein said uncoupling protein is a hybrid protein comprising a heterologous polypeptide sequence that increases the localization of the protein to the mitochondrial membrane.

9. The method of claim 8, wherein said heterologous polypeptide sequence is derived from the yeast ADP/ATP carrier (AAC) protein.

10. The method of claim 1, wherein said screening comprises high-throughput screening.

11. The method of claim 10, wherein said screening comprises robotic high-throughput screening.

12. The method of claim 10, wherein said screening is performed using a multi-well plate.

13. The method of claim 12, wherein said multi-well plate is a 96-well plate.

14. The method of claim 12, wherein said multi-well plate is a 384-well plate.

15. The method of claim 1, wherein said cell is a yeast cell.

16. The method of claim 15, wherein said yeast cell comprises an expression cassette comprising a polynucleotide encoding said uncoupling protein.

17. The method of claim 15, wherein said yeast cell is Saccharomyces cerevisiae.

18. The method of claim 15, further comprising administering to said yeast cell a permeabilizing agent.

19. The method of claim 18, wherein said permeabilizing agent comprises zymolyase.

20. The method of claim 1, wherein said cell is selected from the group consisting of whole untreated cells, permeabilized cells, isolated mitochondria, and proteoliposomes reconstituted with said uncoupling protein.

21. The method of claim 1, wherein said fluorescent probe comprises DiSC3.

22. The method of claim 1, wherein said fluorescent probe is a fluorescent dye other than DiOC6.

23. The method of claim 1, wherein said alteration of said fluorescence comprises an increase or decrease of at least about 30% in the fluorescence intensity in the presence of said test agent compared to the fluorescence intensity in the absence of said test agent.

24. A method of screening a test agent for an ability to modulate uncoupling activity in mitochondria, comprising:
(i) introducing a fluorescent probe into a cell, wherein the fluorescence of said fluorescent probe in said cell is a function of the membrane potential in said mitochondria;
(ii) contacting said cell with said test agent; and
(iii) detecting the fluorescence in said cell;
wherein an alteration in the fluorescence in the cell in the presence of the test agent compared to the fluorescence in the cell in the absence of the test agent indicates an ability of the test agent to modulate uncoupling activity; and
wherein said screening is performed in a homogeneous format.

25. The method of claim 24, wherein said screening comprises high-throughput screening.

26. The method of claim 25, wherein said screening comprises robotic high-throughput screening.

27. The method of claim 25, wherein said high-throughput screening is performed in a multi-well plate.

28. The method of claim 27, wherein said multi-well plate is a 96- or a 384-well plate.

29. The method of claim 24, wherein said cell is a yeast cell.

30. The method of claim 29, wherein said yeast cell is Saccharomyces cerevisiae.

31. The method of claim 29, further comprising administering to said yeast cell a permeabilizing agent.

32. The method of claim 31, wherein said permeabilizing agent comprises zymolyase.

33. The method of claim 24, wherein said cell is selected from the group consisting of whole untreated cells, permeabilized cells, isolated mitochondria, and proteoliposomes.

34. The method of claim 24, wherein said fluorescent probe comprises DiSC3.

35. The method of claim 24, wherein said fluorescent probe is a fluorescent dye other than DiOC6.

36. The method of claim 24, further comprising expressing an uncoupling protein in said cell.

37. The method of claim 36, wherein said uncoupling protein comprises UCP1.

38. The method of claim 36, wherein said uncoupling protein comprises UCP2.

39. The method of claim 36, wherein said uncoupling protein comprises UCP3.

40. The method of claim 36, wherein said uncoupling protein comprises UCP4.

41. The method of claim 36, wherein said uncoupling protein comprises BMCP1.

42. The method of claim 36, wherein said uncoupling protein is a hybrid protein comprising a heterologous polypeptide sequence that increases the localization of the protein to the mitochondrial membrane.

43. The method of claim 42 wherein said heterologous polypeptide sequence is derived from the yeast ADP/ATP carrier (AAC) protein.

44. The method of claim 36, further comprising a secondary screening step wherein the ability of said test agent to modulate uncoupling activity in the absence of said uncoupling protein is assessed, and wherein an ability of said test agent to modulate uncoupling activity in a cell that is expressing said uncoupling protein, but not in a cell that is not expressing said uncoupling protein, indicates that said test agent is specific for said uncoupling protein.

45. The method of claim 24, wherein said alteration of said fluorescence comprises an increase or decrease of at least about 30% in the fluorescence intensity in the presence of the test agent compared to the fluorescence intensity in the absence of the test agent.
